(19) United States
(12) Patent Application Publication
(10) Pub. No.: US 2015/0289782 A1
(43) Pub. Date: Oct. 15, 2015

(54) PORTABLE BREATH VOLATILE ORGANIC COMPOUNDS ANALYSER AND CORRESPONDING UNIT

(71) Applicant: OXFORD MEDICAL DIAGNOSTICS LIMITED, Begbroke Oxon, Oxfordshire (GB)

(72) Inventors: Robert Peverall, Oxon Oxfordshire (GB); Graham Hancock, Oxon Oxfordshire (GB); Grant Andrew Dedman Ritchie, Oxon Oxfordshire (GB)

(73) Assignee: Oxford Medical Diagnostics Limited, Oxon, Oxfordshire (GB)

(21) Appl. No.: 14/442,817

(22) PCT Filed: Nov. 15, 2013
(86) PCT No.: PCT/GB2013/053022

§ 371 Date: May 14, 2015

(30) Foreign Application Priority Data
Nov. 16, 2012 (GB) 1220651.2

(51) Int. Cl. A61B 5/08; G01N 33/497; G01N 1/22; A61B 5/00

(52) U.S. Cl. CPC A61B 5/082; A61B 5/0075; G01N 33/497; G01N 1/22; G01N 2001/2244

(57) ABSTRACT

A compact, portable or handheld device for measurement of breath VOCs such as acetone is described, which incorporates a flow measurement sensor, a mini preconcentrator unit and an spectroscopy unit, such as a cavity-enhanced absorption spectrometer. The preconcentrator includes a chemically selective material to trap VOCs, which is supported on a metal foam. The apparatus is suitable for measuring sub ppm levels of breath VOCs such as acetone and for tracking blood ketone levels.

The present invention relates to a portable, more preferably handheld, analyser apparatus for detecting and quantifying volatile organic compounds (VOCs) in breath, and to a method of detecting and quantifying breath VOCs using such an apparatus. In particular, it can allow the detection and quantification of ketones such as acetone in breath.

It has long been suggested that the level of acetone in exhaled breath, which is a marker of blood ketones, can be used as a possible marker for changing blood glucose levels in type I diabetics. Breath acetone levels are also sensitive to diet and exercise, and thus monitoring them can assist with assessment of diet and exercise regimes.

Type I diabetes sufferers must continually measure their blood glucose levels with checks several times a day. It is also recommended that diabetics who are feeling ill, or those at diabetes onset, also measure their blood ketones in order to prevent diabetic ketoacidosis (DKA)—this is especially relevant for juvenile sufferers. Currently, the most common way of measuring blood glucose levels involves finger lancing and blood testing, and ketones can be measured both by blood and urine testing. However, a non-invasive method for monitoring blood glucose levels and more convenient ways of testing for blood ketones would be extremely useful. Although measurement of breath acetone appears to offer that possibility, current methods of measuring breath acetone rely on mass spectrometry, optical techniques or fuel cell methods, all of which have individual practical difficulties. For example, although mass spectrometric techniques are accurate, they require the use of large and expensive mass spectrometers, and are thus unsuitable for widespread use. Lower-cost techniques of measuring breath acetone have been proposed based on absorption spectroscopy, but these have been too bulky to be realised in a handheld, compact, device. They can also suffer from selectivity problems. For example, the article “A New Acetone Detection Device Using Cavity Ringdown Spectroscopy at 266 nm: Evaluation of the Instrument Performance Using Acetone Sample Solutions” by C Wang and A Mbi (Measurement Science and Technology, 17 Jul. 2007), examines the possibility of using cavity ringdown spectroscopy to measure acetone, but did not produce a compact device and did not operate on breath (instead testing using samples of acetone in deionised water). A later paper measured breath samples indirectly from bags (Wang et al. IEEE SENSORS JOURNAL Volume: 10 Issue: 1 Pages: 54-63 DOI: 10.1109/JSEN.2009.2035730 Published: JAN 2010). More compact methodologies, such as chemical conversion followed by fluorescence spectroscopy, chemical conversion followed by multipass absorption spectroscopy, fuel cell methods or fibre-base spectroscopy suffer from calibration problems or lack of sensitivity.

Thus, although the need for a compact breath VOC analyser has been recognised, none of the currently proposed techniques have delivered one.

Accordingly, the present invention provides a compact, portable analyser apparatus for detecting and quantifying volatile organic compounds (VOCs) in breath in which breath VOCs are adsorbed within an adsorbing material in a preconcentrator and then later released into a compact optical spectroscopic cell. Spectroscopic measurements are then made using emission, fluorescence, impedance or absorption spectroscopy.

The use of the preconcentrator means that the volume of the optical cell can be reduced and the VOC concentration enhanced with simultaneous removal of interfering species (such as water). Thus the volume of the spectroscopic cell is much smaller than the volume of breath collected. This enables the apparatus to be sufficiently compact to be handheld while achieving the required sensitivity of sub ppm levels. In particular, the breath acetone, for example from several hundred cubic centimetres of breath, which is about 30% of a reasonably deep breath, can be efficiently trapped in the adsorbing material and released into a short optical absorption cell with a volume of at most a few cubic centimetres. This allows a volume concentration amplification of one hundred to several hundred times, leading to less stringent sensitivity requirements for the optical cell.

In more detail, the present invention provides a portable analyser apparatus for detecting and quantifying volatile organic compounds in breath, comprising:
a sample inlet for receiving a sample of exhaled breath;
a preconcentrator connected to receive a sample of the breath from the sample inlet and to concentrate volatile organic compounds to form a concentrated sample;
a spectroscopic measurement cell connected to receive the concentrated sample from the preconcentrator and to perform a spectroscopic analysis thereof to detect and quantify volatile organic compounds therein;
a gas handling system for transporting the sample from the sample inlet to the preconcentrator and the concentrated sample from the preconcentrator to the spectroscopic measurement cell and from the spectroscopic measurement cell to an outlet; and
a control system for controlling the gas handling system, the preconcentrator and the spectroscopic measurement cell, and having an output for outputting the spectroscopic analysis result.

The preconcentrator preferably comprises a chemically-selective, preferably hydrophobic, substance for reversibly capturing the VOCs. One suitable type of material is a porous polymer adsorbent in granular or bead form, typically materials used as gas chromatography column fillings, such as Porapak Q. The use of a hydrophobic substance means that water, which is a highly problematic interfering species in breath, tends not to be absorbed, overcoming one of the main problems of spectroscopically analysing breath. The VOC analyte may be a ketone, such as acetone.

Preferably, the chemically-selective substance is held within a metal foam to aid thermal control and increase surface area. The metal foam can, for example, be of an open cell structure porous nickel foam type. The hydrophobic substance may be selected to preferentially absorb the target analyte.

Preferably, the preconcentrator includes a heater, for example, a thin film heater, so that it can be held at a temperature slightly higher than ambient, for example, between 30 and 40° C., or much higher, e.g. 100 to 130° C., as the breath is passed through the preconcentrator.

The gas handling system may include a dry air purge device to purge the preconcentrator with dry air, to remove further water from the sample. The dry air purge device may use a molecular sieve or condenser to dry the air. Alternatively, or in addition, the breath sample may be passed through a chemical trap, or a condenser to chill out water from the breath before the sample passes to the preconcentrator.

The sample inlet may be adapted to allow the subject to exhale directly into it—e.g. by including a mouthpiece, preferably detachable, or being connectable to a mask, which is advantageous in providing a particularly simple and compact apparatus that is easy to use and reduces the possibility of contamination. Alternatively the inlet can be adapted to receive the sample from a receptacle containing the exhaled breath—e.g. a container into which the subject has exhaled and which is then connected to the inlet.

Where the subject exhales into the apparatus directly, the gas handling system preferably includes a flow sensor and controllers to select a desired portion of a stream of breath exhaled into the sample inlet. This allows the apparatus to select a particular portion of the breath, for example two or three hundred cubic centimetres from the end-tidal region of breath. The flow sensor can be, for example, a differential pressure transducer which can be adapted also to record the total volume of exhaled breath. If needed a carbon dioxide sensor can also be incorporated in the apparatus to aid in the breath portioning.

Preferably, the gas handling system further includes a particle filter for filtering the concentrated sample before it is passed to the spectroscopic measurement cell in order to maintain the cleanliness of the cell and to stop particulate matter from entering the optical cell and interfering with the measurements.

Preferably, the spectroscopic measurement cell is an optical cavity for performing cavity-enhanced absorption spectroscopy (CEAS). The CEAS cell may resemble a cylinder with a high reflectivity mirror at either end and input and output ports for introducing and purging the unit of gas samples. The mirrors of the CEAS cell are aligned to form a stable optical cavity. A light source which may be fibre coupled, such as a diode laser, is used to illuminate the input of the CEAS cell, and a photodiode may be used to detect the optical transmission of the cell. The length of the cell should be commensurate with a handheld device, and have an intrinsic sensitivity to acetone of not worse than 100 ppm. The volume of the cell is preferably less than 10 cm³, more preferably less than 2 cm³.

Preferably the analyser apparatus is a handlheld apparatus—the use of the preconcentrator and optical spectroscopy allowing such miniaturisation.

Another aspect of the invention provides a method of detecting and quantifying volatile organic compounds in breath using an analyser in accordance with any one of the preceding claims, the method comprising the steps of:

directing the exhaled breath to the preconcentrator while heating the preconcentrator to a first temperature;

purging the preconcentrator with dry air;

sealing the preconcentrator and heating it to a second temperature higher than the first temperature to release volatile organic compounds;

passing the released volatile organic compounds to the spectroscopic measurement cell and to performing a spectroscopic analysis thereon to detect and quantify the volatile organic compounds; and

purging the preconcentrator while heating it to an elevated temperature to remove any remaining volatile organic compounds.

Preferably, before and/or after the sample has been analysed, the gas handling system is controlled to admit ambient air into the spectroscopic measurement cell so that a background measurement can be made allowing quantification of the VOCs in the sample.

Preferably, the method includes the step, before analysing the concentrated sample, of controlling the gas handling system to select a portion of breath exhaled directly into the inlet and directing it to the preconcentrator.

It is also possible to use the breath acetone measurement made by the analyser to estimate the subject's blood glucose level and preferably this estimation is calibrated by inputting into the analyser a current measurement of the subject's blood glucose level, for example obtained by the conventional blood sample and glucometer method.

The invention will be further described by way of example with reference to the accompanying drawings in which:

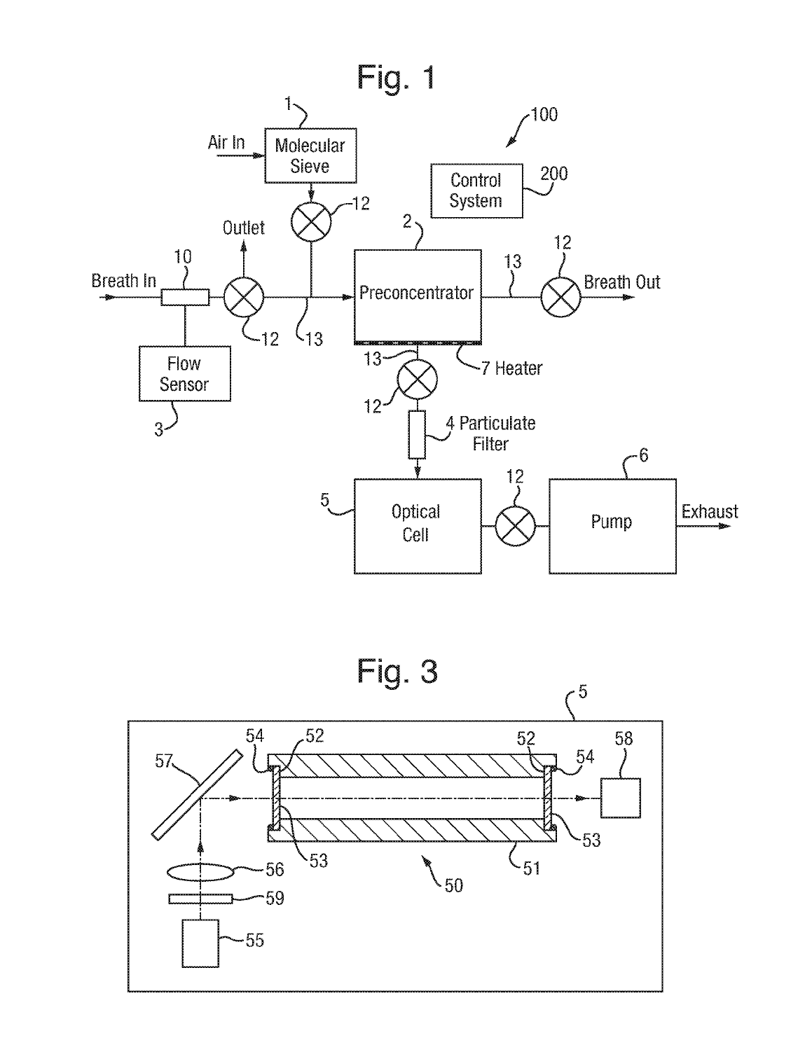

FIG. 1 is a schematic diagram of a handheld breath VOC analyser according to one embodiment of the invention;

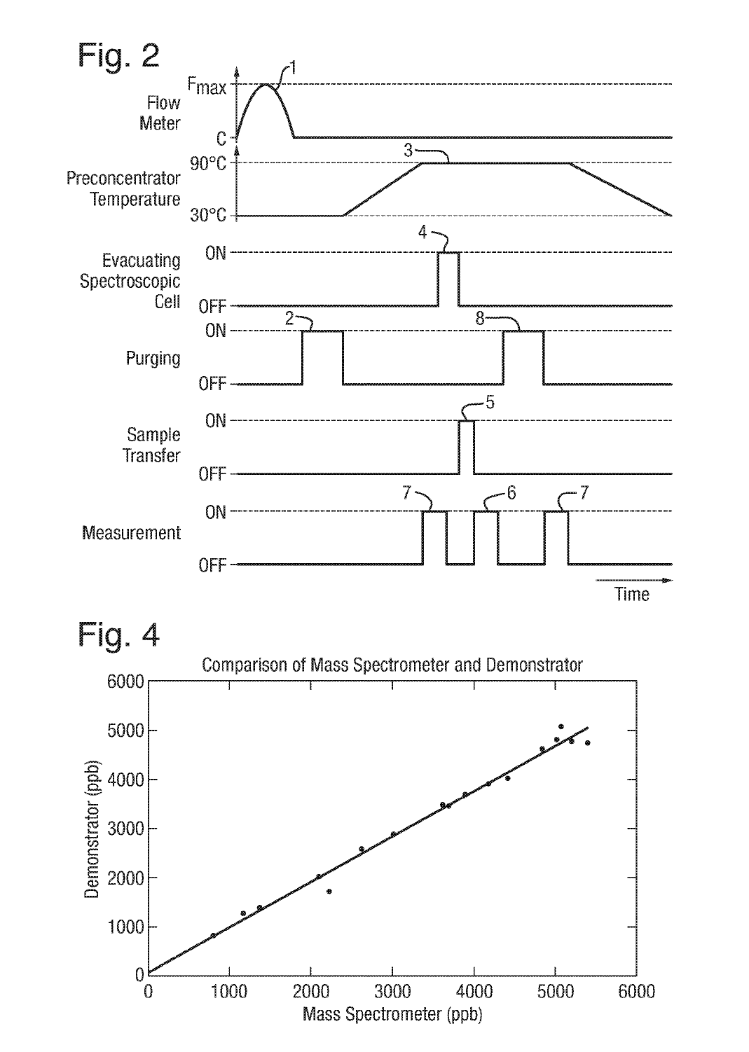

FIG. 2 is a schematic timing diagram of the method of analysis using the analyser of FIG. 1 in one embodiment of the invention;

FIG. 3 is a schematic diagram of the spectroscopic measurement cell in one embodiment of the invention; and

FIG. 4 is a graph comparing the performance of one embodiment of the invention against a mass spectrometer.

As shown in FIG. 1, a handheld breath VOC analyser 100 according to one embodiment of the invention, comprises a sample inlet 10 to which a mouthpiece or mask can be attached to allow a subject to breathe into the device. The analyser 100 includes a gas handling system comprising of a number of valves 12, gas conduits 13, a pump 6 and flow sensor 3 for transporting the sample and also ambient air through the analyser. The various main components of the analyser 100 and the valves 12 are controlled by a control system 200.

In the illustrated embodiment, the gas handling system includes as flow sensor 3 a differential pressure transducer to measure the volume of breath that is exhaled. This quantity is used later for normalisation purposes and in the selection of the portion of exhaled breath that will be passed to the preconcentrator 2. The preconcentrator 2 contains a hydrophobic absorbent material such as Porapak Q, e.g. 0.6 grams, held within a metal, e.g. nickel, foam and also incorporates a thin film heater 7. The heater can be a resistive or Peltier heater, the latter being preferred as it allows active cooling to achieve faster turnaround times between uses. The preconcentrator 2 is preferably as small as possible to reduce the thermal load on the heater. The control system 200 controls the gas handling system to select a certain volume of the breath from which the breath VOCs will be trapped, for example, 200 cubic centimetres from the end-tidal region of breath, this portion of the breath being passed to the preconcentrator 2 with other portions being passed directly out of the analyser 100. The control system, by sensing the gas flow, can detect when the subject is about to end the breath and stop sampling. During the sampling period the heater 7 is used to hold the preconcentrator at a slightly elevated temperature, for example between 30 and 40° C., or higher, e.g. about 130 ° C., as indicated by period (1) in FIG. 2.

When the required volume of breath has been passed to the preconcentrator 2, the preconcentrator 2 is purged with dry air which is pumped into the analyser 100 using a miniature diaphragm pump 6, air being taken from the ambient surroundings and dried using a molecular sieve or condenser device 1 before it passes through the preconcentrator 2. This purging process, represented by period (2) in FIG. 2, reduces the amount of residual water that has been captured by the preconcentrator 2, but has little effect on the trapped VOCs.

In alternative embodiments, residual water can be removed directly from the breath by passing the exhaled breath through a condenser device before it reaches the preconcentrator 2 or by passing the sample through a condenser device or molecular sieve on its way to the optical cell 5.

After several seconds of purging, and as indicated by period (3) in FIG. 2, the preconcentrator 2 is sealed and heated to a higher temperature, for example, about 90° C., by a thin film resistive heater 7 included in the preconcentrator 2. At this temperature, the preconcentrator releases the trapped VOCs which are then passed by the gas handling system to the spectroscopic cell 5 for analysis by first evacuating the spectroscopic cell 5 using pump 6 as indicated by period (4) in FIG. 2, and then opening the spectroscopic cell 5 to the preconcentrator 2 to achieve sample transfer as indicated by period (5).

A particle filter 4 is positioned before the spectroscopic cell 5 to maintain the cleanliness of the cell and to stop particulate matter from entering the cell and interfering with the measurements.

In the preferred embodiment, cavity enhanced absorption spectroscopy is used to measure the VOC level. Where acetone is the target breath analyte, it can be measured using laser or LED sources either in the near infrared (1.6 to 1.8 microns) or UV (230 to 310 nm) spectral regions. For example, a diode laser operating at about 1669-1689, e.g. 1671 nm, or an LED operating at about 275 nm can be used. For use with near infrared wavelengths, the optical cell is constructed with high reflectivity mirrors with reflectivity R>99.95%; and for use with UV wavelengths the mirrors have R>99.6%.

In this embodiment, the volume of the optical cell is less than 10 cm³, more preferably less than 2 cm³, e.g. about 1.5 cm³ , thus providing a volumetric amplification of VOC number density using the preconcentration technique. That is to say, if 200 cm³ of breath passes through the preconcentrator, and all of the target analyte is trapped and then released into the concentrated sample of, say, 5 cm³, a volumetric-driven concentration enhancement factor of 40 is achieved. The absorption reading from the optical cavity is normalised for the volume enhancement.

FIG. 3 schematically illustrates a spectroscopic cell 5 as used in one embodiment of the invention. The optical cell 50 itself is formed from a rigid material (e.g. aluminium) cylinder 51 which has machined into each end shoulders 52 which have a flat surface oriented perpendicular to the longitudinal axis of the cell 51. The cavity mirrors 53, which have complimentary flat peripheral surfaces perpendicular to the optical axis of the mirror, seat against these shoulders ensuring the cell is perfectly aligned and no adjustment is necessary. The cell is also robust and resistant to misalignments caused by physical shock resulting from the portability of the apparatus. A gas tight seal is achieved by the use of o-rings 54.

The light beam from light source 55 is passed through a bandpass filter 59, lens 56 and via a turning mirror 57 into the optical cavity 50. Light exiting the optical cavity 50 is detected by a photodiode 58. The turning mirror 57 is steerable in two dimensions to align the light beam with the optical cavity. Preferably the turning mirror 57 is of the same material as the cavity mirrors. The light source 55, especially when an ultraviolet LED is used, tends to emit a range of frequencies. It is desirable if only those frequencies which have undergone multiple reflection in the optical cavity reach the photodiode 58, otherwise light which is transmitted straight through the cavity mirrors 52 tends to dominate the signal. By making the turning mirror 57 of the same material as the cavity mirrors 52 light to which the mirrors are transparent passes through the turning mirror 57 and does not enter the cavity. The bandpass filter (59) can also be positioned in front of the photodiode (58).

In order to quantify the level of VOCs in the breath, it is necessary to obtain a background measurement of ambient air. As illustrated in period (7) of FIG. 2, such background measurements are preferably taken before and after the sample measurement (6). Thus, for the background measurement, the diaphragm pump 6 is used to admit ambient air through the molecular sieve 1 and into the optical cell 5 for CEAS measurement.

In cavity enhanced absorption spectroscopy (CEAS), the signal (I) and background (Iₒ) are related to the absolute concentration N of analyte in the spectroscopic cell by the equation (Iₒ−I)/I=σNL/(1−R), where σ is the optical absorption cross section at the particular wavelength(s) used, L is the physical length of the cavity within which the sample resides, and R is the geometric mean of the reflectivity of the mirrors. The number density of breath analyte in the subject's breath is therefore N/A where A is the volumetric amplification factor afforded by the instrument. Simplistically, and ignoring any other losses, the amplification factor A linearly depends upon the ratio of the exhaled breath volume to the total cell volume. The sensitivity of CEAS combined with the volumetric amplification resulting from the use of the preconcentrator to supply sample from a larger volume of breath to a small optical cavity allows the detection of sub parts-per-million levels of VOCs to be detected in real time in a compact handheld device. The typical sensitivity achievable for acetone detection should be between 100 and 500 parts per billion.

In the case that the preferred embodiment is for monitoring changes in blood glucose, if needed the central control unit will also accept calibration data from blood glucose measurements such as a finger lance, which may be taken periodically to update the unit's calibration (e.g. once or twice a day), thus allowing a breath acetone measurement to be converted into an estimated blood glucose level. The device may also form part of a general blood glucose or blood ketone management scheme reporting breath acetone and finger lance readings to a central telemedicine hub.

FIG. 4 is a graph comparing the performance of one embodiment of the invention against a mass spectrometer. It shows a plot of breath acetone concentration for breath samples from a volunteer who had undergone various fasting and exercise regimes as measured by an embodiment of the invention and as measured by a mass spectrometer. As can be seen the agreement is good and performance is consistent over a range of breath acetone concentrations from just below 1000 ppb to around 5000 ppb.

1. A portable analyser apparatus for detecting and quantifying volatile organic
compounds in breath, comprising:
a sample inlet for receiving a sample of exhaled breath;
a preconcentrator connected to receive the exhaled breath sample from the sample inlet and to concentrate volatile organic compounds to form a concentrated sample;
a spectroscopic measurement cell connected to receive the concentrated sample from the preconcentrator and to perform a spectroscopic analysis thereof to detect and quantify volatile organic compounds therein;
a gas handling system for transporting the sample from the sample inlet to the preconcentrator and the concentrated sample from the preconcentrator to the spectroscopic measurement cell and from the spectroscopic measurement cell to an outlet; and
a control system for controlling the gas handling system, the preconcentrator and the spectroscopic measurement cell, and having an output for outputting the spectroscopic analysis result.

2. A portable analyser apparatus according to claim 1 wherein the preconcentrator comprises a chemically-selective substance for reversibly capturing the volatile organic compounds.

3. A portable analyser apparatus according to claim 2 wherein the chemically-selective substance is supported by a metal foam.

4. A portable analyser apparatus according to claim 1 wherein the preconcentrator includes a heater.

5. A portable analyser apparatus according to claim 1 wherein the gas handling system includes a dry air purge device to purge the preconcentrator with dry air.

6. A portable analyser apparatus according to claim 5 wherein the dry air purge device comprises one of a molecular sieve or a condenser to dry the air.

7. A portable analyser apparatus according to claim 1 wherein the sample inlet is adapted to receive exhaled breath directly from the subject by the subject exhaling into the inlet.

8. A portable analyser apparatus according to claim 7 wherein the gas handling system includes a flow sensor connected to the sample inlet and means to select a desired portion of a stream of breath exhaled into the sample inlet.

9. A portable analyser apparatus according to claim 1 wherein the sample inlet is adapted to receive exhaled breath from a receptacle.

10. A portable analyser apparatus according to claim 1 wherein the gas handling system includes a particle filter for filtering the concentrated sample before it is passed to the spectroscopic measurement cell.

11. A portable analyser apparatus according to claim 1 wherein the spectroscopic measurement cell is an optical cavity for performing cavity-enhanced absorption spectroscopy.

12. A method of detecting and quantifying volatile organic compounds in breath using an analyser in accordance with anyone of the preceding claims, the method comprising the steps of:
directing the exhaled breath to the preconcentrator while heating the preconcentrator to a first temperature;
purging the preconcentrator with dry air;
sealing the preconcentrator and heating it to a second temperature higher than the first temperature to release volatile organic compounds;
passing the released volatile organic compounds to the spectroscopic measurement cell and to performing a spectroscopic analysis thereon to detect and quantify the volatile organic compounds; and
purging the preconcentrator while heating it to an elevated temperature to remove any remaining volatile organic compounds.

13. A method according to claim 12 further comprising the step, before and/or after analysing the concentrated sample, of controlling the gas handling system to admit ambient air into the spectroscopic measurement cell and spectroscopically analysing the ambient air.

14. A method according to claim 12 further comprising the step of inputting to the control system a measurement of the subject's blood glucose level, calibrating the spectroscopic quantification of the volatile organic compounds in the subject's breath against the inputted blood glucose level, whereby further measurements of the quantity of volatile organic compounds in the subject's breath provide an estimate of the subject's blood glucose level.

15. A method according to claim 12, further comprising the step, before analysing the concentrated sample, of controlling the gas handling system to select a portion of breath exhaled directly into the inlet and directing it to the preconcentrator.

16. A portable analyser apparatus according to claim 3 wherein the preconcentrator includes a heater.

17. A portable analyser apparatus according to claim 3 wherein the gas handling system includes a dry air purge device to purge the preconcentrator with dry air.

18. A portable analyser apparatus according to claim 6 wherein the sample inlet is adapted to receive exhaled breath directly from the subject by the subject exhaling into the inlet.

19. A portable analyser apparatus according to claim 6 wherein the sample inlet is adapted to receive exhaled breath from a receptacle.

20. A portable analyser apparatus according to claim 8 wherein the gas handling system includes a particle filter for filtering the concentrated sample before it is passed to the spectroscopic measurement cell.
